(19) United States
(12) Patent Application Publication
(10) Pub. No.: US 2025/0049438 A1
(43) Pub. Date: Feb. 13, 2025

(54) DEVICE FOR PERFORMING ANASTOMOSIS

(71) Applicants: IMPLICAN B.V., Groningen (NL); RIJKSUNIVERSITEIT GRONINGEN, Groningen (NL); ACADEMISCH ZIEKENHUIS GRONINGEN, Groningen (NL)

(72) Inventors: Klaas Havenga, Groningen (NL); Ivo Kooijman, Groningen (NL)

(21) Appl. No.: 18/696,055

(22) PCT Filed: Sep. 28, 2022
(86) PCT No.: PCT/NL2022/050542

§ 371 Date: Mar. 27, 2024

(30) Foreign Application Priority Data
Sep. 28, 2021 (NL) 2029264

(51) Int. Cl. A61B 17/11; A61B 17/00; A61B 90/00

(52) U.S. Cl. CPC A61B 17/1114; A61B 90/03; A61B 2017/00477; A61B 2017/1132; A61B 2090/034

(57) ABSTRACT

A kit of parts is provided for forming a device for performing anastomosis a first gastro-intestinal tract. The kit of parts comprises an inner ring, comprising an outer surface generally facing away from the passage, and an outer ring, comprising an inner surface. When the inner ring and the outer ring are in an assembled state, the inner ring is at least partially positioned in the reception volume of the outer ring, at least part of the outer surface of the inner ring faces towards the inner surface of the outer ring, and the outer surface and the inner surface together define an annular space between them, wherein at least part of the annular space converges in the generally axial direction.

### TECHNICAL FIELD

The aspects and embodiments thereof relate to the field of devices for performing anastomosis, in particular of two gastro-intestinal tract sections.

### BACKGROUND

Anastomoses, i.e. the joining of two sections of tubular organ sections of the gastro-intestinal tract such as the oesophagus, colon or other parts, are frequently associated with complications such a leakage, infections, fibrosis and the like. For instance, anastomotic leakage is observed in about 10% of low anterior resection cases involving anastomosis of the colorectal segment by conventional circular staplers.

Favourable results in terms of preventing complications after anastomosis have been obtained by using compression anastomosis (see e.g. Kaider-Person et al., The American Journal of Surgery (2008) 195, 818-826). Conventional compression anastomosis includes for instance clamping and fixating the two tissue layers of the gastro-intestinal tract between two members of a clamping device such as the known Murphy button, Boerema knot, Valtrac™ or magnetic rings (see Cossu et al., The American Surgeon (2000) (8), 759-762 and Jansen et al. Surgery, Gynecoloy & Obstetrics (1981) 153, 537-545 respectively).

NL2017917 discloses a method and a surgical instrument, typically a surgical stapler that is configured for performing anastomosis of a first gastro-intestinal tract section to a second gastro-intestinal tract section, both gastro-intestinal tract sections comprising a superficial layer and an intermediate layer. Said method comprises contacting the superficial layers of said gastro-intestinal tract sections and compressing the contacted sections between a first pressure area and a second pressure area, wherein the sum of the first pressure area and the second pressure increases during the compression such that the superficial layers are pressed aside and the intermediate layers are contacted.

US20110264121A1 discloses a sleeve type fixing method for anastomosis for tubular organs. Two intestines are overlapped in an anastomosis portion between an inner ring and an outer ring. A width of the anastomosis portion is about 4-5 millimetres, which allows the serous membranes of the intestines to be contacted with each other.

U.S. Pat. No. 5,290,298 discloses a fragmentable compression device for the anastomosis of hollow organs of the human body.

### SUMMARY

In spite of the favourable results, compression anastomosis is still posed by challenges and therefore the use of conventional surgical staplers is generally standard in anastomosis procedures. Conventional surgical staplers give no compression on the connected bowel segments and preserve the circulation of blood between the staples. It is accordingly desirable to further improve compression anastomosis so it will i.a. become easier to perform and more accessible. It is desired to improve compression anastomosis in terms of the healing process such to reduce post-operational complication.

A first aspect provides a kit of parts for forming a device for performing anastomosis of a first gastro-intestinal tract section and a second gastro-intestinal tract section. The kit of parts comprises an inner ring, defining a passage for faecal matter through the inner ring in a generally axial direction, and comprising an outer surface generally facing away from the passage, an outer ring, defining a reception volume for receiving at least part of the inner ring therein, the outer ring comprising an inner surface facing at least partially towards the reception volume, wherein the inner ring and the outer ring are arranged to, in an assembled state in which the inner ring is at least partially positioned in the reception volume of the outer ring, have at least part of the outer surface of the inner ring facing towards the inner surface of the outer ring, and to have the outer surface and the inner surface together define an annular space between them, wherein at least part of the annular space converges in the generally axial direction. It will be appreciated that an annular space converging in a first axial direction is the same as the annular space diverging in a second axial direction opposite to the first axial direction.

In general, when the inner ring and the outer ring are referred to as being in assembled state, this may refer to a situation wherein parts of the first gastro-intestinal tract section and the second gastro-intestinal tract section are present between the inner ring and the outer ring, in particular in the annular space.

At least one of the inner ring and the outer ring may be deformed when the first gastro-intestinal tract section and the second gastro-intestinal tract section are present between the inner ring and the outer ring. As such, the shape of the annular space may be different in situation with and without the gastro-intestinal tract sections present between the inner ring and outer ring.

In order to be deformed, at least one of the inner ring and the outer ring may comprise a resilient, an elastic and/or a deformable material. In particular, at least one of the outer surface of the inner ring and the inner surface of the outer ring may be deformable.

The tissue of the gastro-intestinal tract is a tubular layered structure basically comprising three layers of different tissue types. The peripheral layer primarily comprises muscularis, the subsequent submucosal layer primarily comprises collagen and the luminal layer primarily comprises mucosa.

The annular space may be arranged for accommodating part of both the first gastro-intestinal tract section and the second gastro-intestinal tract section. In particular, the annular space may be sized to accommodate the submucosal layers of both the first gastro-intestinal tract section and the second gastro-intestinal tract section.

When, in use, the kit of parts is assembled by positioning the inner ring at least partially in the reception volume of the outer ring, the first gastro-intestinal tract section and the second gastro-intestinal tract section may become compressed in the annular space between the inner ring and the outer ring. The present inventors found that sufficient compression causes the peripheral layer and the luminal layer to become thinner, in particular even up to the point of the peripheral layer and the luminal layer becoming cut off—leaving only or substantially only the submucosa layer present in at least part of the annular space.

Compression of the gastro-intestinal tract sections may be achieved by virtue of the annular space between the inner ring and the outer ring being insufficiently large to accommodate the full thickness of the first gastro-intestinal tract section and the second gastro-intestinal tract section combined in uncompressed state. Because in assembled state, at least part of the annular space converges in the generally axial direction, different amounts of compression can be applied to the first gastro-intestinal tract section and the second gastro-intestinal tract section.

The amount of compression may increase in the generally axial direction, in particular in a downstream direction of the gastro-intestinal tract. As such, when regarded in an upstream direction, the amount of compression may at some point reach or generally be zero. This may hold true for one or both gastro-intestinal tract sections, and/or for one or more layers of a gastro-intestinal tract section such as the submucosa layer.

A thickness of at least part or a majority of the annular space in a radial direction perpendicular to the axial direction may lie between 0.1 and 1.0 mm, and in particular between 0.15-0.75 mm or even between 0.3-0.5 mm.

A thickness of at least part or even a majority of the annular space in a radial direction perpendicular to the axial direction may be 1.0 mm or smaller, 0.5 mm or smaller, or even 0.3 mm or smaller. It has been observed that when the thickness of the annular space in the radial direction is 1.0 mm or smaller, in particular even 0.5 mm or smaller, submucosa layers of the first gastro-intestinal tract section and the second first gastro-intestinal tract section can be placed in contact with each other, which in turn may advantageously result in healing by primary intent between the submucosa layers. It will be understood that although part of the annular space may have a width smaller than 1.0 mm, another part of the annular space may have a width larger than 1.0 mm, and the annular space converges from this larger width to the width smaller than 1.0 mm. The desired thickness of the annular space in the radial thickness typically depends on the wall thickness of the gastro-intestinal tract sections, in particular on a thickness of the submucosa layers.

In particular, as the width of the annular space reduces to 1.0 mm or smaller, 0.5 mm or smaller, or even 0.3 mm or smaller, one or both of the serosa and mucosa layers—i.e. respectively peripheral layers and luminal layers—of the gastro-intestinal tract sections may be peeled or stripped off the submucosal layer as the inner ring is moved into the reception volume of the outer ring. Additionally or alternatively, by virtue of the reduced thickness in the annular space, one or both of the serosa and mucosa layers may be crushed or disintegrated, thereby exposing the submucosal layer.

Because the inner ring and the outer ring are arranged to, in an assembled state, have an annular space defined between them, another part of the annular space may have a thickness of more than 0.5 mm or more than 1.0 mm. As the width of the annular space reduces, in use when the inner ring is moved further into the outer ring, the submucosa layers of the first gastro-intestinal tract section and the second first gastro-intestinal tract section are placed in contact with each other.

It has been observed that the contact between the submucosa layers of the first gastro-intestinal tract section and the second gastro-intestinal tract section may improve healing between the first gastro-intestinal tract section and the second gastro-intestinal tract section and/or may improve stability of the anastomosis in the gastro-intestinal tract section of the patient, for example compared to a situation wherein only serosa layers of both gastro-intestinal tract sections are contacted. It has been observed that the submucosa layer forms a backbone of the gastro-intestinal tract section, and thus an anastomosis between the submucosa layers of the gastro-intestinal tract sections may be preferred—as an alternative to or an addition to an anastomosis between serosa layers and/or mucosa layers.

Preferably, but not necessarily, the submucosa layers are also contacted outside the outer ring, for example axially outside the outer ring, for example above or below the outer ring. Outside the outer ring may imply outside a reception volume of the outer ring, and/or outside an inner diameter of the outer ring.

It will be understood that one or both of the inner ring and the outer ring may be deformed, elastically and/or plastically, when the inner ring and the outer ring are moved into the assembled state with the gastro-intestinal tract sections positioned in-between the inner ring and the outer ring. As such, the desired shape and dimensions of the annular space may in examples only be achieved when the gastro-intestinal tract sections are positioned in the annular space. It will hence be understood that wherever in the present disclosure the inner ring and the outer ring are said to be in the assembled state, this can imply that the gastro-intestinal tract sections are positioned in the annular space.

Thus, when in the assembled state parts of two gastro-intestinal tract sections are positioned in the annular space, a thickness of at least part of the annular space in a radial direction perpendicular to the axial direction may be 0.5 mm or smaller, and in particular 0.3 mm or smaller, such that in this assembled state submucosal layers of the gastro-intestinal tract sections can be contacted.

As a further option applicable to any kit of parts disclosed herein, in the assembled state, a thickness of the annular space perpendicular to a centreline of the annular space may be 1.0 mm or smaller for part of the annular space, in particular 0.5 mm or smaller or even 0.3 mm or smaller, and 1.0 mm or larger, or even 0.5 mm or larger for another part of the annular space. These particular thicknesses allows the gastro-intestinal tract sections to be pressed together such that the submucosal layers are contacted.

As such, it will be understood that any kit of parts disclosed herein, comprising any inner ring and any outer ring, in any combination, may be arranged such that in the assembled state with parts of two gastro-intestinal tract sections positioned in the annular space, a thickness of the annular space perpendicular to a centreline of the annular space is 1.0 mm or smaller for part of the annular space, in particular 0.5 mm or smaller, 0.3 mm or smaller, and 0.5 mm or larger or even 1.0 mm or larger for another part of the annular space, such that in assembled state submucosal layers of the gastro-intestinal tract sections can be contacted.

The direction for passage of faecal matter through the inner ring may be defined generally as an axial direction, wherein faecal matter typically moves in a downstream direction through the gastro-intestinal tract. The outer surface of the inner ring facing away from the passage for faecal matter is thus oriented at least partially in a radial direction, wherein a radial direction is defined as being perpendicular to the axial direction.

In general, the inner ring may be approximately symmetric, in particular rotationally symmetric, around a centreline of the inner ring and the outer ring may be approximately rotationally symmetric, in particular rotationally symmetric, around a centreline of the outer ring. In use, in assembled state, the centrelines of the inner ring and the outer ring may be aligned or at least approximately aligned. A ring such as the inner ring and/or the outer ring may have a generally circular or donut-like shape, or may have any other shape such as generally oval or elliptical.

At least one of the outer surface of the inner ring and the inner surface of the outer ring may be provided by a rigid material comprised by the respective at least one of the inner ring and the outer ring.

In general, the term rigid is used to imply that in use of the device for performing anastomosis, a material comprised by the device does not deform in any degree which may impact the usability of the device. Common rigid materials are metals, composite materials, and thermosetting polymers.

The opposite of a rigid material is a resilient material. The term resilient material implies that in use of the device for performing anastomosis, the material comprised by the device may be deformed when a force in an order typical for the use of the device is applied to the material. The material thus exhibits a particular stiffness, which may be expressed in N/mm—i.e. the amount of force in Newtons required for a particular deformation expressed in millimetres. When the force is no longer applied to the material, the material may at least partially elastically deform back into its original shape. As such, in an elastically deformed state, the resilient material may provide a force in a direction opposite to the direction of the force applied to it.

As an option, the annular space between the outer surface of the inner ring and the inner surface of the outer ring may comprise, next to the diverging part, a non-diverging part with a substantially constant cross-sectional area in a plane perpendicular to the axial direction.

A height of the annular space in a direction parallel to the axial direction may be between 4-20 mm, in particular between 4-6 mm, or at least larger than 2 mm or at least larger than 4 mm. This height may allow for a sufficient part of the first gastro-intestinal tract section and the second gastro-intestinal tract section to become compressed in the annular space.

Different types of connections may be used for connecting the inner ring and the outer ring. It is preferred to connect the inner ring and the outer ring to each other and to the gastro-intestinal tract during or after their placement to prevent migration of one or both rings through the gastro-intestinal tract, for example due to peristaltic movements or the passage of faecal matter through the rings.

In general, the connection between the inner ring and the outer ring may be a clamped connection, for example using an interference fit between both rings, a snap-fit connection such as a cantilever snap-fit, comprising one or more interlocking components, a friction connection, any other connection, or any combination thereof. For a clamped connection, parts of one or both of the inner ring and the outer ring may be resilient.

A particular snap-fit connection may be obtained when part of a first of the inner ring and the outer ring has a larger footprint than part of a second of the inner ring and the outer ring. The footprint may be regarded in a top view, perpendicular to a centreline of the inner ring and the outer ring. The part with the larger footprint may hook behind the part with a smaller footprint, when the inner ring is at least partially positioned in the reception volume of the outer ring. A ring with an ellipsoid inner and/or outer shape may have a larger footprint than a ring with a circular inner and/or outer shape.

A second aspect provides a method for performing anastomosis of a first gastro-intestinal tract section and a second gastro-intestinal tract section. The method comprises the steps of positioning an inner ring in the first gastro-intestinal tract section and at least partially enclosing the inner ring in the first gastro-intestinal tract section, positioning an outer ring in the second gastro-intestinal tract section and at least partially enclosing the outer ring in the second gastro-intestinal tract section, and moving the inner ring in an axial direction into a reception volume of the outer ring. After the inner ring is moved sufficiently into the reception volume of the outer ring, the inner ring may be or become connected to the outer ring.

During the moving of the inner ring into the reception volume of the outer ring, an annular space is formed between an outer surface of the inner ring and an inner surface of the outer ring, which annular space converges in the generally axial direction, such that part of the first gastro-intestinal tract section and part of the second gastro-intestinal tract section are compressed together in the annular space.

The compressed parts of the first gastro-intestinal tract section and part of the second gastro-intestinal tract section are generally oriented in the axial direction, because at least part of the annular space may also be generally oriented in the axial direction.

In particular, after the inner ring is moved into the reception volume of the outer ring, primarily submucosal layers of at least part of the first gastro-intestinal tract section and part of the second gastro-intestinal tract section are present in the annular space. Being primarily present may imply that a significant portion, for example more than 50% or even more than 80% of the volume of the part of the annular space filled with the gastro-intestinal tract is filled with the submucosal layers.

The peripheral layer and the luminal layer may be compressed more than the submucosal layer for example due to the material composition of the different layers. The materials comprised by the submucosal layer may have a higher stiffness against compression, and the thickness of the submucosal layer may be reduced less under compression than the peripheral layer and the luminal layer.

It is preferred that when the two gastro-intestinal tract sections are compressed between the inner ring and the outer ring, the submucosal layers of the two gastro-intestinal tract sections become placed in contact with each other. In general, by contacting the two submucosal layers, healing by primary intent may be promoted. Healing by primary intent may increase the healing process rate and reduce the risk of leakage, infections, fibrosis and the like. The contact between the two submucosal layers may be achieved in the annular space, or outside of the annular space.

Additionally, or alternatively, matter such as fluids may be pressed or squeezed out of one or both of the peripheral layer and the luminal layer, causing the thickness and/or volume of these two layers to become reduced under compression. Additionally, or alternatively, one or both of the peripheral layer and the luminal layer may be peeled or stripped off the submucosal layer, in particular when the inner ring is moved into the reception volume of the outer ring.

When the two gastro-intestinal tract sections are compressed between the inner ring and the outer ring, debris, bacteria, or any other matter which is inside the lumen of the gastro-intestinal tract sections may be pushed or moved towards the centreline of the gastro-intestinal tract sections, which may correspond to the passage for faecal matter through the inner ring. As such, it may be at least partially prevented that the debris, bacteria, or any other matter contacts a healing area where the two submucosal layers contact each other.

During the moving of the inner ring into the reception volume of the outer ring, outer ends of the first gastro-intestinal tract section and the second gastro-intestinal tract section may be cut off. The cut off parts may exit the body via the anus. Cutting of the gastro-intestinal tract sections may be performed using two interacting parts of the inner ring and the outer ring. One or both of the interacting parts may have one or more sharp or serrated edges or surfaces against the other of the interacting parts may for example be pressed. Parts of the gastro-intestinal tract section positioned between the two interacting parts may as such be fully or partially cut, or at least perforated.

In embodiment of the method, an anvil with a cutting edge may be used for cutting parts of the gastro-intestinal tract sections. The anvil is a separate element from the inner ring and the outer ring. In use, the anvil may be positioned inside the inner ring. When the inner ring with the anvil is positioned inside the first gastro-intestinal tract section, the anvil may be used to pull the inner ring into the reception volume of the outer ring. When the inner ring cannot move further into the reception volume of the outer ring, the anvil may disconnect from the inner ring and may be pulled through the rings, thereby cutting part of the first and second gastro-intestinal tract section enclosing respectively the inner ring and the outer ring.

As an option, an auxiliary holding ring may be comprised by the kit of parts, which is arranged to cooperate with the anvil in cutting the gastro-intestinal tract sections. In use, the auxiliary holding ring may be positioned downstream of the outer ring in the second gastro-intestinal tract section. The auxiliary holding ring may comprise a sharp and/or serrated surface and/or edge. After the anvil passes through the inner ring, the anvil may interface with the sharp or serrated portion of the auxiliary holding ring for cutting the gastro-intestinal tract sections.

In embodiments of the method, after the inner ring is moved into the reception volume of the outer ring, the submucosal layers of the two gastro-intestinal tract sections become placed in contact with each other, in particular over the full circumference. By virtue of the shape of the annular space, the contacting parts of the submucosal layers may be essentially isolated from bacteria originating from mucosa and faecal matter. It may be preferred to provide contact between the muscularis layers of the two gastro-intestinal tract sections outside but near the annular space, as it may promote healing by primary intent.

A third aspect provides a placement device for placing a device for performing anastomosis of a first gastro-intestinal tract section and a second gastro-intestinal tract section comprising an inner ring and an outer ring.

It will be appreciated that the method according to the second aspect may be performed using components of a kit of parts according to the first aspect and/or using a placement device according to the third aspect. As such, optional features disclosed in conjunction with one of the aspects may be readily applied to embodiment of the other two aspects.

### BRIEF DESCRIPTION OF THE FIGURES

In the figures,

FIG. 1A shows a top view next to a perspective view of a first embodiment of a kit of parts;

FIG. 1B shows the cross-section A-A as indicated in the top view of FIG. 1A;

FIG. 2 schematically shows the kit of parts of FIGS. 1A and 1B in an unassembled state;

FIGS. 3A, 3B, and 3C show different stages of a method for performing anastomosis of a first gastro-intestinal tract section and a second gastro-intestinal tract section;

FIG. 4A, FIG. 4B and FIG. 4C show a second embodiment of a kit of parts for forming a device for performing anastomosis;

FIGS. 5A and 5B show two perspective views of a placement device for placing a device for performing anastomosis of two gastro-intestinal tract sections;

FIG. 6A shows an embodiment of part of a placement device;

FIGS. 6B and 7A-7D show different section views of a placement device;

FIG. 8 shows a detailed view of FIG. 3C;

FIG. 9 shows a colour photo of an experiment in which two gastro-intestinal tract sections have been compressed into an annular space between an outer ring and an inner ring; and

FIG. 10 shows a black-and-white version of the photo of FIG. 9.

### DETAILED DESCRIPTION OF THE FIGURES

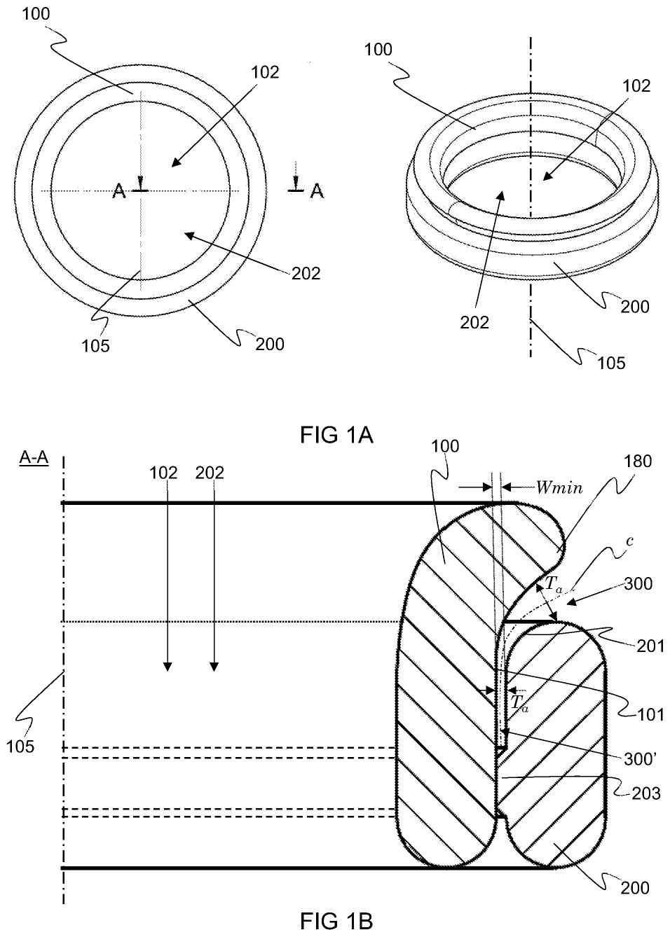

FIG. 1A shows a top view next to a perspective view of a first embodiment of a kit of parts for forming a device for performing anastomosis of a first gastro-intestinal tract section and a second gastro-intestinal tract section, in an assembled state. FIG. 1B shows the cross-section A-A as indicated in the top view of FIG. 1A.

The kit of parts comprises an embodiment of an inner ring 100, defining a passage 102 for faecal matter therethrough. The inner ring 100 comprises an outer surface 101 generally facing away from the passage 102.

The embodiment of the kit of parts as depicted in FIGS. 1A and 1B further comprises an outer ring 200, defining a reception volume 202 for receiving at least part of the inner ring therein. The outer ring 200 comprises an inner surface 201, facing at least partially towards the reception volume 202.

In the assembled state, for example depicted in FIG. 1B, the inner surface 201 of the outer ring 200 faces towards the inner ring 100. The outer surface 101 of the inner ring 100 faces towards the outer ring 200. In particular, the outer surface 101 at least partially faces the inner surface 201.

The outer ring 200 and inner ring 100 are as an option positioned coaxially relative to centreline 105, and substantially rotationally symmetric around centreline 105. In FIGS. 1A and 1B, an axial direction may be defined being parallel to the centreline 105.

In the assembled state, for example depicted in FIG. 1B, an annular space 300 is formed between the outer ring 200 and the inner ring 100, in particular between the outer surface 101 and the inner surface 201.

As shown in FIG. 1B, as an option, the outer ring 200 may comprise an inner flange 203 protruding towards or even into the passage 102 for faecal matter. In particular, the inner flange 203 may have a smaller inner diameter than one or more sections of the inner surface 201 of the outer ring 200 adjacent to the inner flange 203.

An inner diameter of the inner flange 203 may correspond to an outer diameter of part of the outer surface 101 of the inner ring 100. To correspond here may imply one of an interference fit, light interference fit, or a loose fit between the inner flange 203 and the inner ring 100. One or both of the faces adjoining the inner flange 203 with the adjacent parts of the inner surface 201 of the outer ring may be rounded-off or bevelled, or at least partially oriented at an angle relative to the radial direction.

Although the inner flange 203 is in FIGS. 1A and 1B depicted as being comprised by the outer ring 200, it will be appreciated that embodiments of the inner ring 100 may have an outer flange in addition to or as an alternative to the inner flange 203.

When a fit such as an interference fit is achieved between the inner ring 100 and the outer ring 200, in any embodiment of the kit of parts, for example by virtue of the inner flange 203, leakage of faecal matter between the inner ring 100 and the outer ring 200 may be reduced or even prevented.

An option, shown in FIG. 1B, and applicable to any embodiment of the kit of parts, may be that part 300′ of the annular space 300 has an approximately constant cross-section. A radial thickness of this part 300′ may correspond to the thickness of two compressed submucosal layers.

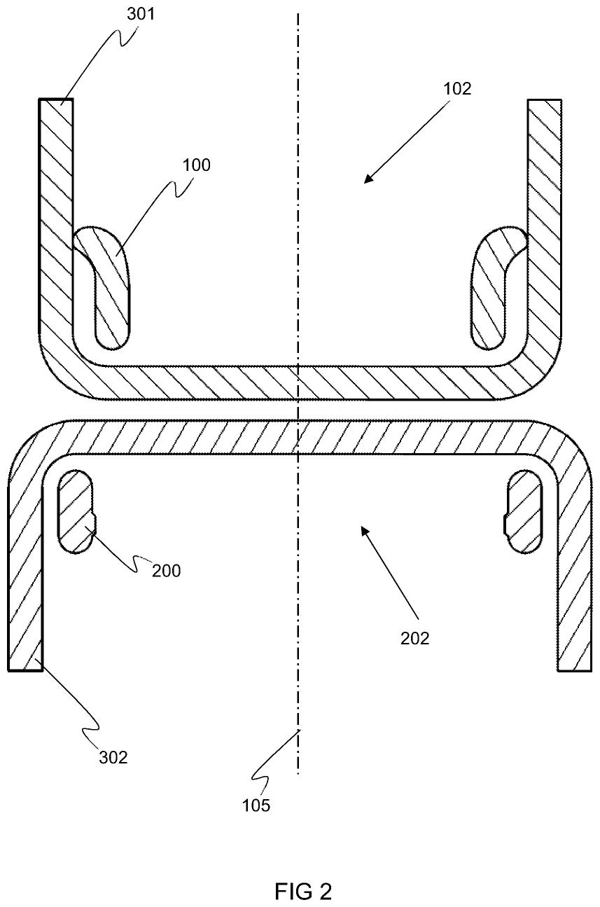

FIG. 2 schematically shows the kit of parts of FIGS. 1A and 1B in an unassembled state. The inner ring 100 is positioned in a first gastro-intestinal tract section 301, which may be a proximal gastro-intestinal tract section. The outer ring 200 is positioned in a second gastro-intestinal tract section 302, which may be a distal gastro-intestinal tract section. The gastro-intestinal tract sections may be at least partially closed using for example a purse string suture, and may as such partially enclose their respective gastro-intestinal tract section.

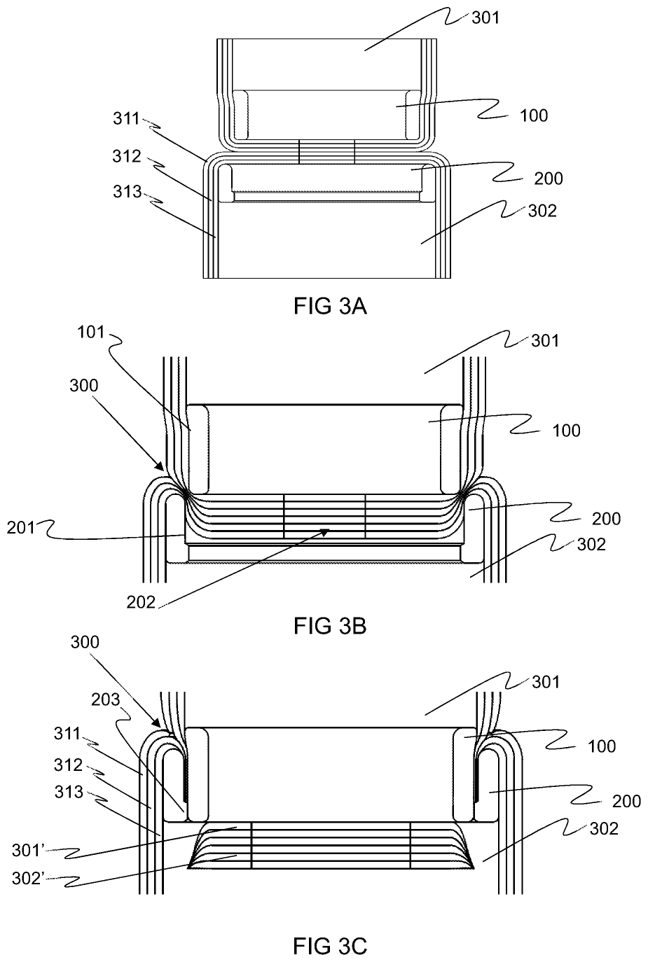

FIGS. 3A, 3B, and 3C show different stages of a method for performing anastomosis of a first gastro-intestinal tract section 301 and a second gastro-intestinal tract section 302. As schematically depicted in FIGS. 3A, 3B, and 3C, a gastro-intestinal tract section generally comprises a peripheral layer 311 comprising muscularis, a luminal layer 313 comprising mucosa and a submucosal layer 312 between the peripheral layer 311 and the luminal layer 313.

In a first method step depicted in FIG. 3A, an embodiment of the inner ring 100 is positioned in the first gastro-intestinal tract section 301, and an embodiment of the outer ring 200 is positioned in the second gastro-intestinal tract section 302. The gastro-intestinal tract sections at least partially enclose respectively the inner ring and the outer ring, as can also be seen in FIG. 2. As such, for example when the inner ring 100 is moved axially, the first gastro-intestinal tract section 301 moves axially with the inner ring. The inner ring and the outer ring may be arranged to slide or telescope one into the other.

In a second method step depicted in FIGS. 3B and 3C, the inner ring 100 is at least partially moved axially into the reception volume 202 of the outer ring 200. During this movement, the annular space 300 is formed between the outer surface 101 of the inner ring 100 and the inner surface 201. Part of the first gastro-intestinal tract section 301 and part of the second gastro-intestinal tract section 302 become compressed into the annular space 300, which annular space 300 may thus have a radial thickness smaller than the thicknesses of the two gastro-intestinal tract sections.

FIG. 3C shows a third method step wherein part of the first gastro-intestinal tract section and part of the second gastro-intestinal tract section are compressed together in the annular space. Furthermore, another part of the first gastro-intestinal tract section 301′ and another part of the second gastro-intestinal tract section 302′ have been cut off, for example while moving the inner ring 100 into the outer ring 200. The cut-off parts of the first gastro-intestinal tract section 301′ and the second gastro-intestinal tract section 302′ may have enclosed respectively the inner ring 100 and the outer ring 200 prior to the moving of the inner ring 100 in the axial direction into the reception volume 202 of the outer ring 200.

The parts of the gastro-intestinal tract sections 301′ and 302′ may be cut off by virtue of the fit between the inner flange 203 of the outer ring 200 and the inner ring 100, or by means of a different cutting portions comprised by one or both of the inner ring and the outer ring.

As schematically represented in FIG. 3C, primarily the submucosal layer 312 is present in the annular space 300, for example by virtue of the particular radial thickness of the annular space 300. In the annular space 300, the peripheral layer 311 and/or the luminal layer 313 may be compressed further than the submucosal layer 312.

In general, by virtue of the diverging, tapered, and/or funnelled shape of the annular space, different amounts of compression of the gastro-intestinal tract may be achieved in different sections of the gastro-intestinal tract. For example, in or near a widest part of the annular space, the gastro-intestinal tracts may be exposed to little to no compression, whereas in the more narrow parts of the annular space, the compression increases. At or near a part of the annular space where the gastro-intestinal tracts are exposed to little to no compression, the uncompressed submucosa layers may be approximated. Further towards the more narrow parts of the annular space, the two submucosa layers may be gradually compressed.

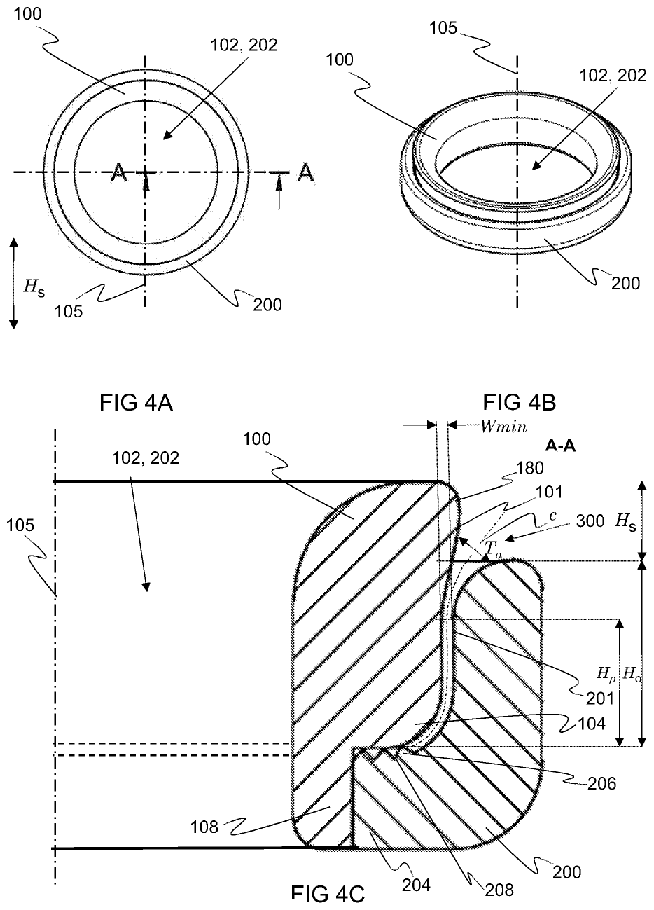

FIG. 4A, FIG. 4B and FIG. 4C show a second embodiment of a kit of parts for forming a device for performing anastomosis, respectively in a top view, a perspective view, and a cross-sectional view along plane A-A depicted in FIG. 3A.

The second embodiment of the kit of parts comprises an inner ring 100 and an outer ring 200, wherein the inner ring 100 defines a passage 102 for faecal matter and the outer ring defines a reception volume 202 for receiving at least part of the outer ring 200.

The kit of parts is in FIG. 4A, FIG. 4B and FIG. 4C depicted in an assembled state. As such, an annular space 300 is defined between an outer surface 101 of the inner ring 100, and an inner surface 201 of the outer ring 200. Part of the annular space 300 converges in the generally axial direction, parallel to the centreline 105, in particular in the downstream axial direction. In other words, the annular space 300 diverges in the upstream axial direction.

As a particular option, for example shown in FIG. 4C, the outer ring comprises an inwards shoulder section 204 as an example of an inner flange. The inner ring 100 comprises an outward shoulder section 104. In the assembled state, the inward shoulder section 204 is arranged to abut the outward shoulder section 104, in particular on a plane which is oriented at an angle relative to the centreline 105, for example approximately perpendicular to the centreline 105—i.e. in the radial direction.

As an even further option, for example shown in FIG. 4C, the inward shoulder section 204 of the outer ring 200 is provided with one or more serrations 206 protruding from a surface 208 of the inward shoulder section, which surface 208 faces at least partially in the axial direction—i.e. parallel to the centreline 105. By virtue of the serrations 206, the gastro-intestinal tract sections may be cut or at least perforated.

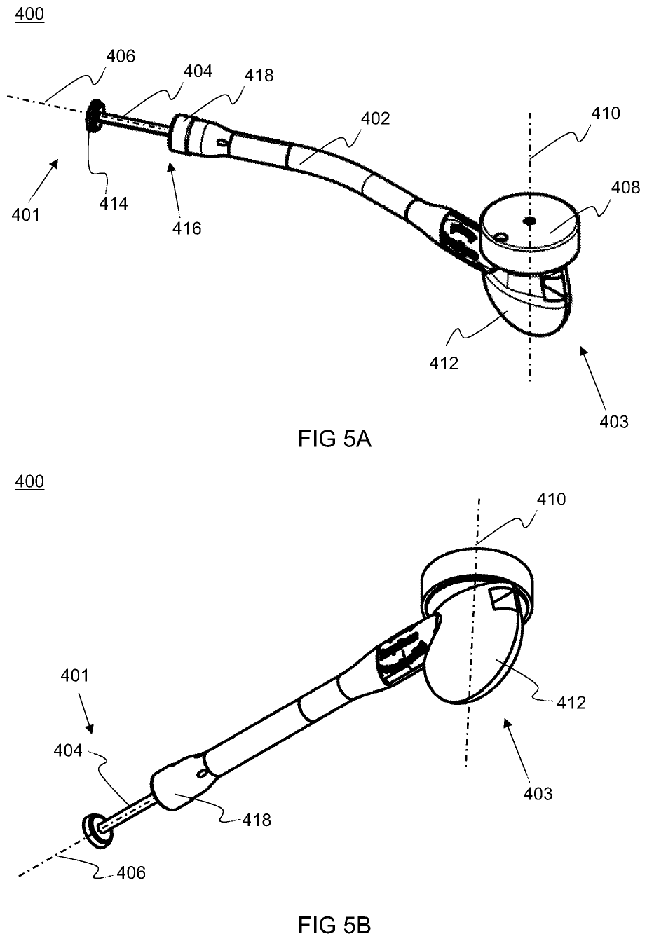

FIGS. 5A and 5B show two perspective views of a placement device 400 for placing a device for performing anastomosis of two gastro-intestinal tract sections.

The placement device 400 comprises a housing 402, which may a flexible housing in the sense that a surgeon may at least partially manipulate the shape of the housing 402. For example, the housing 402 may comprise multiple sections which may be hingedly or rotatably connected such that the sections can be hinged or rotated relative to each other.

At a distal end 401 of the housing 402, the placement device 400 comprises a translatable element 404 arranged to be connected to the inner ring of the device for performing anastomosis, and which translatable element is arranged to be translated over a translation axis 406. At a distal end of the translatable element 404, a connection element 414 may be provided for connection with the inner ring. The connection element 414 may be a thickened element relative to the translatable element 404—i.e. the connection element 414 may have a larger outer diameter than the translatable element 404.

At a proximal end 403 of the housing, the placement device 400 comprises an actuator 408 arranged to be rotated around a rotation axis 410, which rotation axis is positioned at an angle relative to the translation axis 406. In particular, the rotation axis 410 may be positioned perpendicular to the translation axis 406. For example, when part of the housing 402 is flexible, the translation axis 406 and the rotation axis 410 do not necessarily intersect.

For connecting the actuator 410 to the translatable element 404, the placement device further comprises a coupling mechanism arranged for converting a rotation of the actuator to a translation of the translatable element, for example using a rack and pinion mechanism, threaded spindle, a cycloid transmission, or any other threaded mechanism. The coupling mechanism may be positioned in and/or covered by the housing 402, and is hence not visible in FIGS. 5A and 5B. An actuator shall be understood as a component arranged to receive a force and/or torque input, for example from a surgeon. An actuator may also be referred to as an actuatable element.

A transmission may be comprised by the coupling mechanism, for example comprising one or more gears, racks, or any other component for transferring a rotation. The transmission may be used to convert a rotational speed of the actuator into a lower speed of the translatable element. This in turn may decrease the torque required for rotating the actuator. For example, the transmission may have a gear ratio of 32:1 or more, 16:1 or more, or 2:1 or more.

As an option, the placement device 400 further comprises a handle 412, which may be positioned opposite to the actuator 408. As such, the axis of rotation 410 may extend through part of the handle 412. The handle 412 is connected to the housing at the proximal end 403 of the housing.

At least part of the handle 412 may be shaped as at least part of an ellipsoid, in particular a triaxial ellipsoid, and in particular as more than halve of an ellipsoid. As such, the handle 412 may be conveniently held in hands of different sizes.

In use of the device, the placement device 400 may be inserted into the patient at the distal end 401, for example via the anus of the patient. In use, the proximal end 403 of the placement device 400 may be positioned outside the body of the patient, for example such that the actuator 408 may be actuated by a surgeon.

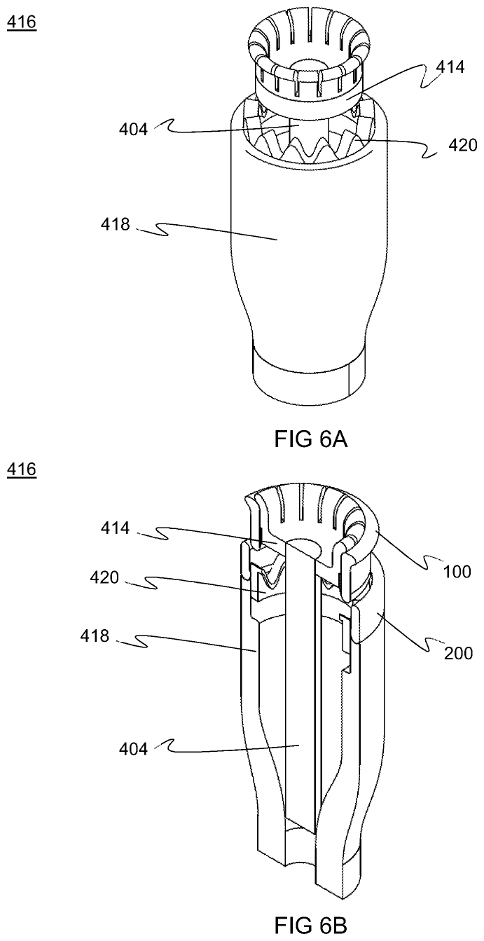

FIG. 6A shown in a perspective view an embodiment of an end-effector 416 of a placement device, for example a placement device as depicted in FIG. 5A. The end-effector 416 comprises the translatable element 404 and a connection element 414 for connection with an inner ring.

FIG. 6B schematically shows the end-effector 416 of FIG. 6A in a section view, with an inner ring 100 connected to the connection element 414 and an outer ring 200 connected to an end-effector body 418. As can be seen in FIG. 6B, by moving the translatable element 404 and with that the connection element 414, the inner ring 100 can be moved towards and into the outer ring 200.

The placement device as an option comprises a cutting ring 420, which may for example comprise a sharp cutting edge or a serrated end section as shown in FIG. 6A. By virtue of the serrations of the serrated end section, parts of the gastro-intestinal section may be cut-off. The outer ring 200 may be fixated to the cutting ring 420, and may as such be fixated to the end-effector 416 of the placement device.

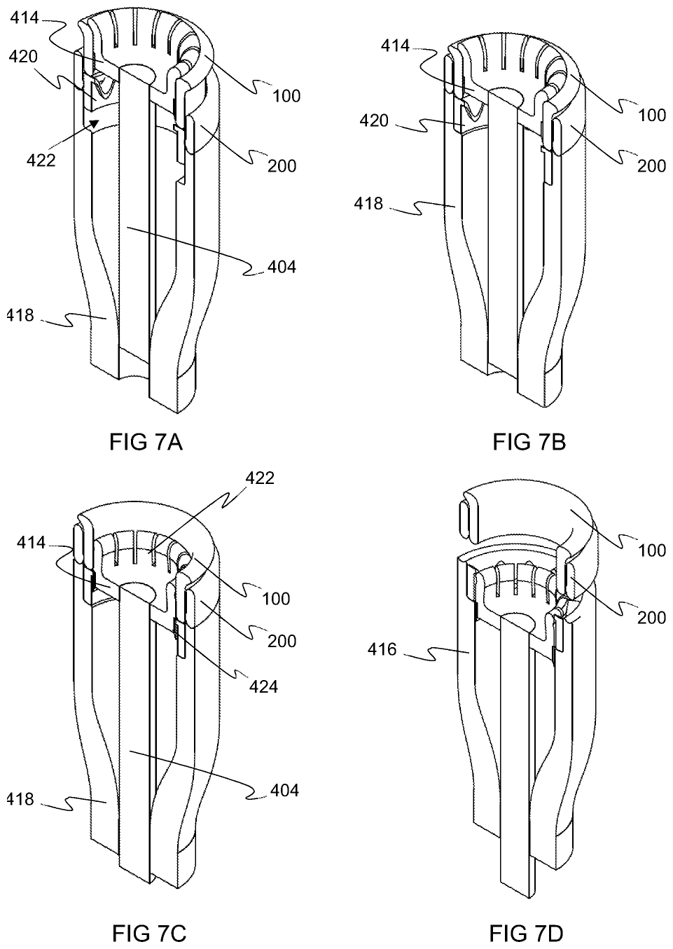

FIGS. 7A-7D schematically depict four steps of a method of placing an inner ring 100 and an outer ring 200 for performing an anastomosis. For clarity of the figures, the gastro-intestinal tract is not depicted.

In a first step depicted in FIG. 7A, the inner ring 100 is partially positioned in the outer ring 200, and contacts the cutting ring 420. By pulling the translatable element 404 further down in a second step, the situation of FIG. 7B is obtained. As can be seen in FIG. 7B, the cutting ring 420 has moved relative to the end-effector body 418, which has an optional reception volume 422 for accommodating movement of the cutting ring 420. Prior to the first step, the inner ring has been positioned in the first gastro-intestinal tract section, and the outer ring has been positioned in the second gastro-intestinal tract section, as for example depicted in FIG. 3A.

In the situation of FIG. 7B, the annular space between the outer surface of the inner ring and the inner surface of the outer ring has been completely formed.

FIG. 7C depicts a third step wherein the translatable element 404 is pulled even further down. As a particular option, the connection element 414 to which the inner ring 100 is connected comprises a flexible crown 422. The flexible crown 422 in the first and second step allows the inner ring 100 to be pulled down into the outer ring 200. The amount of force required for pulling the inner ring 100 down may increase as the inner ring 100 is moved further into the outer ring 200, up to a point in which the flexible crown 422 flexes inward, thereby sliding through the inner ring 100.

The flexible crown 422 may for example comprise a plurality of hinging fingers as shown in FIGS. 7A-7D, or in general may comprise any elastic or flexible material which allows deformation of the flexible crown 422. The deformation may be elastic and/or plastic—for example, one or more fingers may break away to allow passage of the connection element 414 through the inner ring 100.

Between the situations depicted in FIGS. 7B and 7C, as the inner ring connection element 414 passes through the inner ring 100, a cutting edge 424 of the connection element 414 may interact with the cutting ring 420 to cut parts of the gastro-intestinal tract section. The connection element 414 may as such act as a cutting anvil.

FIG. 7D depicts a fourth step in which the end-effector 416 has been pulled away from the inner ring and the outer ring. This allows the end-effector 416 to be removed from the body of the patient, leaving behind the inner ring and the outer ring, with the two gastro-intestinal tract sections compressed between the inner ring and the outer ring.

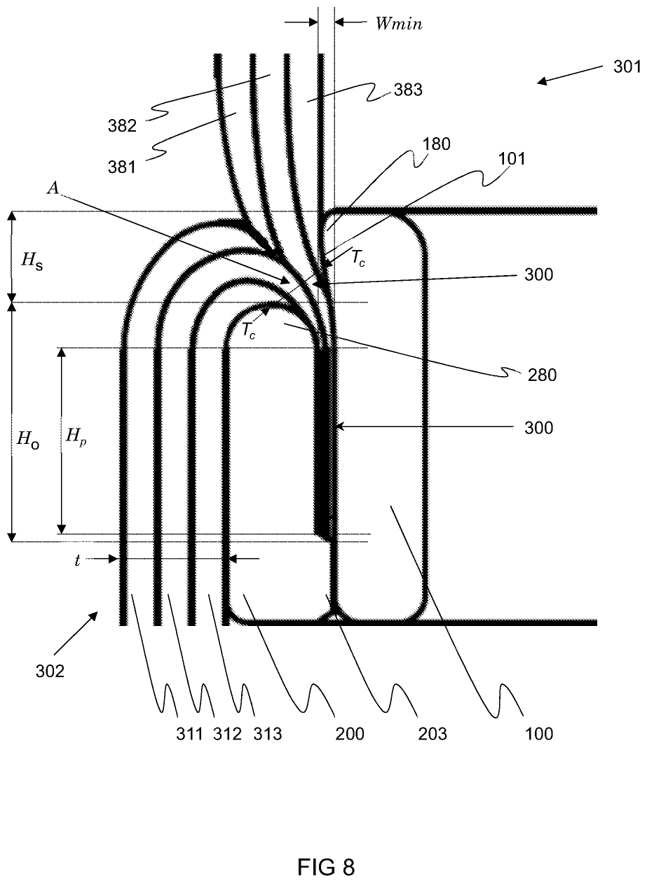

FIG. 8 shows a detailed view of the third method step wherein part of the first gastro-intestinal tract section 301 and part of the second gastro-intestinal tract section 302 are compressed together in the annular space 300 between the inner ring 100 and the outer ring 200. FIG. 8 is a detailed view of part of FIG. 3C.

As an option shown in FIG. 8 but also in other figures, and readily applicable to any embodiment of the inner ring 100 disclosed herein, the inner ring 100 may have an outward protruding shoulder 180. Part of the outer surface 101 of the inner ring 100 is formed by the shoulder 180, which part may in use contact the luminal layer 383 of the first gastro-intestinal tract section 301. Generally, any shoulder disclosed herein may alternatively be referred to as a flange, or radially extending portion.

Although in the example of FIG. 8, the shoulder 180 does not extend radially beyond an inner diameter of the outer ring 200, in other examples of the inner ring 100, the shoulder 180 may extend further radially. For example, an outward protruding shoulder 180 of the inner ring 100 may protrude beyond the inner surface of the outer ring 200 (see e.g. the example of FIG. 1B). In other examples, the outward protruding shoulder 180 of the inner ring 100 may even protrude beyond at least part of the outer surface of the outer ring 200 or even beyond the outer diameter of the outer ring 200.

The inventors have found that particular dimensions of the inner ring 100 and outer ring 200 may be beneficial to the healing process between the gastro-intestinal tract sections and/or the stability of the inner ring and the outer ring in the gastro-intestinal tract and/or the strength of the anastomosis. In particular, dimensions of the inner ring 100 and the outer ring 200 are preferably chosen such that in use submucosal layers 312 and 382 of the gastro-intestinal tract sections are contacted. A contact surface area between the submucosal layers 312 and 382 may be created or enlarged by virtue of several dimensions of the inner ring and the outer ring.

Preferably, a minimal thickness, also referred to as width Wₘᵢₙ, of part of the annular space 300 is 0.5 mm or smaller, in particular even 0.3 mm or smaller. This minimal thickness is preferably achieved over a certain height, in FIG. 8 denoted as Hₚ. This height Hₚ may be between 1.0-5.0 mm, in particular between 1.0-2.0 mm. Over the height Hₚ, the width of the annular space 300 may be essentially constant. A further part of the annular space may have an even smaller width than the width Wₘᵢₙ, for example by virtue of the inner flange 203 of the outer ring 200. It has been observed that a height Hₚ between 1.0-5.0 mm prevented slippage of the gastro-intestinal tract sections out of the annular space between the inner ring and the outer ring.

In the example of FIG. 8, the width Wₘᵢₙ is achieved over a section of the annular space 300 which is parallel to the axial direction. It will however be understood that embodiments of the inner ring and outer ring are envisioned wherein the part of the annular space 300 over which the width Wₘᵢₙ is achieved is oriented at an angle relative to the axial direction. The minimal width or minimal thickness Wₘᵢₙ may in such cases be regarded in a direction normal to the outer surface of the inner ring, normal to the inner surface of the outer ring, or normal to both when said outer surface and said inner surface are oriented parallel.

An axial height of the outer ring 200 contributing to the annular space 300 is in FIG. 4C and FIG. 8 denoted as Hₒ. When in the assembled state part of the inner ring 100, such as but not limited to the outward protruding shoulder 180, axially extends beyond the outer ring 200, the total height Hₒ+Hₛ of the annular space 300 may be further defined by the inner ring 100. Preferably, the height Hₒ is between 2.0-6.0 mm, in particular between 2.0-3.0 mm, and the height Hₛ is between 1.5-2.5 mm, in particular between 1.75-2.25 mm, in any combination thereof.

By virtue of the annular space 300 converging in the generally axial direction, in particular in a downward direction, a combined wall thickness of the two gastro-intestinal tract sections, regarded in a direction normal or perpendicular to at least one of the gastro-intestinal tract sections, referred to as thickness T_{c}, is gradually reduced as the tract sections are compressed in the annular space 300 when the inner ring is moved into the outer ring. In particular, the thickness T_{c} is reduced from maximally the sum of the wall thicknesses t of the two gastro-intestinal tract sections in uncompressed state, to the minimal width Wₘᵢₙ, or even further for example where the gastro-intestinal tract sections are cut—see for example cut-off sections 301′ and 302′ in FIG. 3C. The sum of the wall thicknesses of the gastro-intestinal tract sections in uncompressed state is generally referred to as Tmax, with Tmax generally equal to two times t. It will be understood that for different humans the wall thickness of the gastro-intestinal tract—indicated in FIG. 8 as thickness t—may differ.

For example, typically, the wall thickness of the gastro-intestinal tract is approximately 2.25 mm, or between 2.0 and 2.5 mm, for a colon of an adult human. Depending on the age and physical condition of a human, the wall thickness of a gastro-intestinal tract may even be between 1.5 and 3.0 mm. It will be understood however that due to a medical condition, and/or a particular treatment such as radiation treatment, a wall thickness of the gastro-intestinal tract may exceed 3.0 mm. As such, a kit of parts may be formed using an inner ring and an outer ring specifically sized to form an annular space with a thickness Tₐ perpendicular to a centreline of part of the annular space being 1.5 mm or smaller, 1.0 mm or smaller, 0.5 mm or smaller, in particular between 1.5 mm and 0.05 mm, depending on the wall thicknesses of the gastro-intestinal tract sections on which the anastomosis has to be performed.

The skilled person will appreciate that typically, a gastro-intestinal tract section may have lobes and/or sections which are folded over itself. As such, the wall thicknesses defined herein may apply to gastro-intestinal tract section which are stretched out, for example by virtue of an inner ring or outer ring being inserted into the gastro-intestinal tract sections. Typically, the outer diameter of the outer ring can range between 20 mm and 40 mm, in particular between 25 mm-35 mm.

It will be understood that the aspects and embodiments of the present disclosure may be used for anastomosis of any type of gastro-intestinal tract sections, such as but not limited to an oesophagus, intestines, small intestine, large intestine, and any other part of the gastro-intestinal tract.

It has been observed that between Tmax and Wₘᵢₙ, an optimal or at least advantageous spot or range—also referred to as a sweet spot or sweet range—can be found. At this spot or in this range, it has been found that healing by primary intent takes place between the submucosal layers of the gastro-intestinal tract sections. In particular, the optimal or at least advantageous thickness T_{c}, with Wₘᵢₙ<T_{c}<Tmax, perpendicular to at least one of the gastro-intestinal tract sections lies between 0.2 mm and 3.5 mm.

It is hence preferred that at least part of the annular space is sized such that in the assembled state, with parts of two gastro-intestinal tract sections positioned in the annular space, the gastro-intestinal tract section are compressed to a thickness between 0.2 mm and 3.5 mm, in particular between 0.5 mm and 2.5 mm.

As best depicted in FIG. 8, at least part of the contacting area between the submucosal layers 312, 382 may be positioned outside the annular space 300. Additionally or alternatively, at least part of the contacting area between the submucosal layers 312, 382 may be positioned above the outer ring when regarded in an axial direction. This contacting area is generally indicated with arrow A in FIG. 8.

For any annular space of any kit of parts comprising an inner ring and outer ring disclosed herein, a centreline can be defined. For example in FIGS. 1B, 4C, and 10, the centreline is indicated as a dash-dotted line c. From any point on the centreline, a distance towards the inner surface of the outer ring and a distance towards the outer surface of the inner ring, perpendicular to the centreline, is equal. The sum of these two distances is referred to as the thickness Tₐ of the annular space 300. Examples of the thickness Tₐ are indicated in FIGS. 1B, 4C, 9 and 10. The thickness Tₐ in assembled state of the inner ring and the outer with parts of two gastro-intestinal tract sections positioned in the annular space correspond to the thickness T_{c}, i.e. the combined thicknesses of the compressed gastro-intestinal tract sections.

In use, when the two gastro-intestinal tract sections are present in the annular space 300, the contact area A between the two gastro-intestinal tract sections may approximately follow this centreline, in particular when a stiffness of the gastro-intestinal tract sections is comparable. In practical examples, a deviation between this contact area A and the centreline in at least part of the annular space 300 between the two gastro-intestinal tract sections may for example be ±10% or even ±20% or more.

In any embodiment of the kit of parts comprising any inner ring and any outer ring disclosed herein, in any combination, a thickness Tₐ of at least part of the annular space 300 perpendicular to the centreline c may range between 0.0 mm and 10.0 mm or even between 0.0 mm and 5.0 mm. Preferably, the thickness Tₐ—defined perpendicular to the centreline c—of at least part of the centreline c

It will be appreciated that when at least one of the outer ring and the inner ring is deformed in assembled state with the gastro-intestinal tract sections in the annular space, the shape of the centreline c and the thickness Tₐ at different positions along the centreline c may be affected by the presence of the gastro-intestinal tract sections in the annular space.

As for example visible in FIG. 8, part of the second gastro-intestinal tract section 302 is bent radially inward and protrudes into the annular space. Part of the second gastro-intestinal tract section 302 in the annular space can be regarded as being inverted relative to the rest of the second gastro-intestinal tract section 302. In particular, part of the second gastro-intestinal tract section 302 is bent over the outer ring 200, over a curved upper portion 280 of the outer ring 200. A curvature of said upper portion 280 may be designed such to achieve a desired shape of the converging part of the annular space 300.

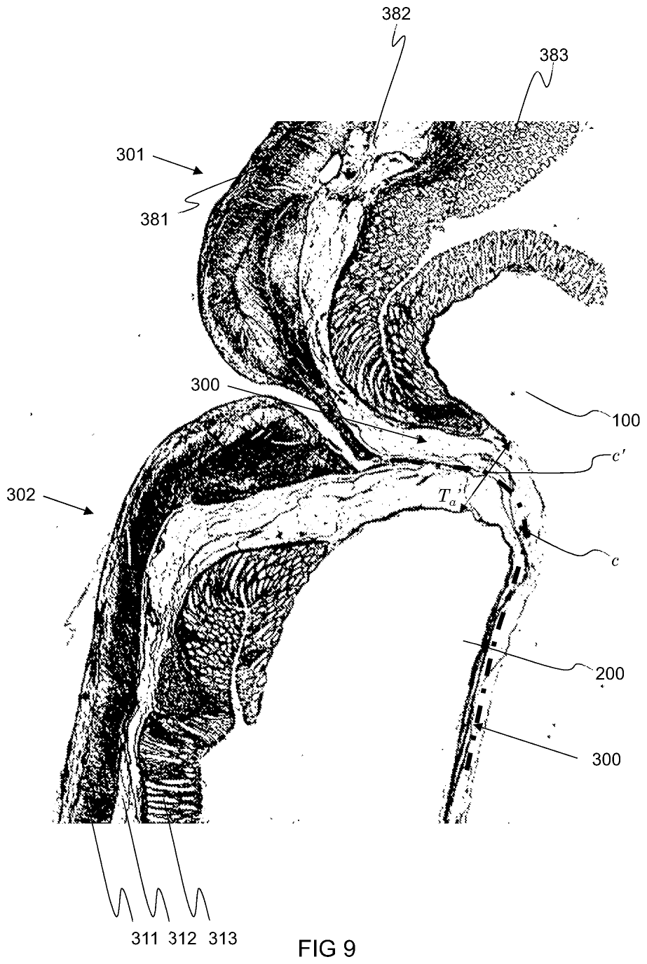

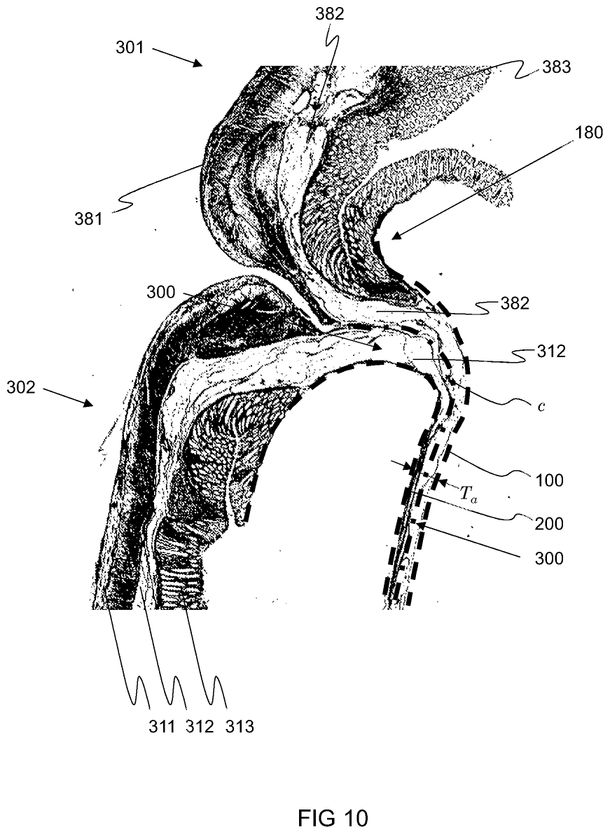

FIG. 9 shows a photo of an experiment in which two gastro-intestinal tract sections 301, 302 have been compressed into an annular space 300 formed by moving an inner ring 100 into an outer ring 200. FIG. 10 shows the same photo, albeit in black-and-white whereas FIG. 9 shows a coloured photo. FIGS. 9 and 10 clearly depict the different layers comprised by the gastro-intestinal tract sections 301, 302—i.e. the peripheral layers 311, 381, submucosal layers 312, 382, and the luminal mucosa layers 313, 383.

In FIG. 9, the shape of the inner ring and outer ring can be identified using the shape of the deformed gastro-intestinal tract sections 301, 302. To further clarify the shape of the inner ring 100 and the outer ring 200, the contours of the rings are schematically drawn in FIG. 10 as dashed lines. The inner ring and outer ring shown in assembled state in FIGS. 9 and 10 form the annular space, which has a thickness Tₐ perpendicular to the centreline c of 1.0 mm or smaller for part of the annular space, and 1.0 mm or larger for another part of the annular space.

As depicted in FIG. 9, at a particular point c′ on the centreline c of the annular space 300, the submucosal layers 312, 382 are contacted because the peripheral layers 311, 381 have been pressed away, thereby exposing the submucosal layers. The particular point c′ on the centreline has an associated thickness Tₐ′, which may range between 0.5 mm and 3.5 mm, in particular between 1.0 mm and 2.5 mm.

FIGS. 9 and 10 also shows that by virtue of the size and shape of the annular space 300, the luminal layers 313 and 383 have been stripped or torn off the submucosal layers, or at least have been crushed or compressed away. As such, in at least part of the annular space, in particular at least part of the converging part of the annular space, essentially only the submucosal layers are present, or at least a majority of said part of the annular space is occupied by submucosal layers.

In the description above, it will be understood that when an element is referred to as being connect to another element, the element is either directly connected to the other element, or intervening elements may also be present. Also, it will be understood that the values given in the description above, are given by way of example and that other values may be possible and/or may be strived for.

It is to be noted that the figures are only schematic representations of embodiments that are given by way of non-limiting examples. For the purpose of clarity and a concise description, features are described herein as part of the same or separate embodiments, however, it will be appreciated that the scope of the disclosure may include embodiments having combinations of all or some of the features described.

The word ‘comprising’ does not exclude the presence of other features or steps. Furthermore, the words ‘a’ and ‘an’ shall not be construed as limited to ‘only one’, but instead are used to mean ‘at least one’, and do not exclude a plurality.

1. A kit of parts for forming a device for performing anastomosis of a first gastro-intestinal tract section and a second gastro-intestinal tract section, the kit of parts comprising:
an inner ring, defining a passage for faecal matter through the inner ring in a generally axial direction, and comprising an outer surface generally facing away from the passage;
an outer ring, defining a reception volume for receiving at least part of the inner ring therein, the outer ring comprising an inner surface facing at least partially towards the reception volume;
wherein the inner ring and the outer ring are arranged to, in an assembled state in which the inner ring is at least partially positioned in the reception volume of the outer ring, have at least part of the outer surface of the inner ring facing towards the inner surface of the outer ring, and to have the outer surface and the inner surface together define an annular space between them, wherein at least part of the annular space converges in the generally axial direction.

2. The kit of parts according to claim 1, wherein in the assembled state, a thickness of at least part of the annular space in a radial direction perpendicular to the axial direction is 1.0 mm or smaller.

3. The kit of parts according to claim 1, wherein in the assembled state with parts of two gastro-intestinal tract sections positioned in the annular space, a thickness of at least part of the annular space in a radial direction perpendicular to the axial direction is 1.0 mm or smaller, such that in assembled state submucosal layers of the gastro-intestinal tract sections can be contacted.

4. The kit of parts according to claim 1, wherein in the assembled state, a thickness of the annular space perpendicular to a centreline of the annular space is 1.0 mm or smaller for part of the annular space, and 1.0 mm or larger for another part of the annular space.

5. The kit of parts according to claim 1, wherein in the assembled state with parts of two gastro-intestinal tract sections positioned in the annular space, a thickness of the annular space perpendicular to a centreline of the annular space is 1.0 mm or smaller for part of the annular space, and 1.0 mm or larger for another part of the annular space, such that in assembled state submucosal layers of the gastro-intestinal tract sections can be contacted.

6. The kit of parts according to claim 1, wherein at least one of the outer surface and the inner surface is provided by a rigid material comprised by the respective at least one of the inner ring and the outer ring.

7. The kit of parts according to claim 1, wherein the annular space between the outer surface of the inner ring and the inner surface of the outer ring comprises a non-diverging part with a constant cross-sectional area in a plane perpendicular to the axial direction.

8. The kit of parts according to claim 1, wherein a height of the annular space in a direction parallel to the axial direction is between 2-20 mm.

9. The kit of parts according to claim 1, wherein the outer ring comprises an inner flange protruding towards the passage for faecal matter.

10. The kit of parts according to claim 9, wherein an inner diameter of the inner flange corresponds to an outer diameter of part of the outer surface of the inner ring.

11. The kit of parts according to claim 1, wherein the outer ring comprises an inward shoulder section, the inner ring comprises an outward shoulder section, and in the assembled state, the inward shoulder section is arranged to abut the outward shoulder section.

12. The kit of parts according to claim 11, wherein the inward shoulder section of the outer ring is provided with one or more serrations protruding from a surface of the inward shoulder section, which surface faces at least partially in the axial direction.

13. The kit of parts according to claim 1, wherein the inner ring comprises an outward protruding shoulder.

14. The kit of parts according to claim 13, wherein in the assembled state, at least part of the outward protruding shoulder axially extends beyond the inner ring.

15. The kit of parts according to claim 1, wherein the outer ring has a shorter height than the inner ring when an end of the inner ring is aligned with an end of the outer ring.

16. A method for performing anastomosis of a first gastro-intestinal tract section and a second gastro-intestinal tract section, the method comprising:
positioning an inner ring in the first gastro-intestinal tract section and at least partially enclosing the inner ring in the first gastro-intestinal tract section;
positioning an outer ring in the second gastro-intestinal tract section and at least partially enclosing the outer ring in the second gastro-intestinal tract section; and
moving the inner ring in an axial direction into a reception volume of the outer ring;
wherein during the moving of the inner ring into the reception volume of the outer ring, an annular space is formed between an outer surface of the inner ring and an inner surface of the outer ring, which annular space converges in the generally axial direction, such that part of the first gastro-intestinal tract section and part of the second gastro-intestinal tract section are compressed together in the annular space.

17. The method according to claim 16, wherein after the inner ring is moved into the reception volume of the outer ring, primarily submucosal layers of part of the first gastro-intestinal tract section and part of the second gastro-intestinal tract section are present in the annular space.

18. The method according to claim 16, wherein during the moving of the inner ring into the reception volume of the outer ring, outer ends of the first gastro-intestinal tract section and the second gastro-intestinal tract section are cut off.

19. The method according to claim 16, wherein after the inner ring is moved into the reception volume of the outer ring, the submucosal layers of the two gastro-intestinal tract sections become placed in contact with each other.

20. A placement device for placing a device for performing anastomosis of a first gastro-intestinal tract section and a second gastro-intestinal tract section comprising an inner ring and an outer ring, the placement device comprising:
a housing;
at a distal end of the housing, a translatable element arranged to be connected to the inner ring of the device for performing anastomosis, and which translatable element is arranged to be translated over a translation axis; and
at a proximal end of the housing, an actuator arranged to be rotated around a rotation axis, which rotation axis is positioned at an angle relative to the translation axis, in particular perpendicular;
wherein the placement device further comprises a coupling mechanism connecting the actuator to the translatable element, which coupling mechanism is arranged for converting a rotation of the actuator to a translation of the translatable element.

21. (canceled)

22. (canceled)
